(19) United States
(12) Patent Application Publication
(10) Pub. No.: US 2010/0305082 A1
(43) Pub. Date: Dec. 2, 2010

(54) Use of FKBPL gene to identify a cause of infertility

(76) Inventors: Stephen Downes, Coleraine (GB); Colum Walsh, Coleraine (GB); Olaf Sunnotel, County Antrim (GB); Laszlo Hiripi, Godollo (HU); David Hirst, Belfast (GB); Tracy Robson, Belfast (GB)

Correspondence Address: GREENLEE SULLIVAN P.C., 4875 PEARL EAST CIRCLE, SUITE 200, BOULDER, CO, 80301, US

(21) Appl. No.: 12/808,139

(22) PCT Filed: Dec. 15, 2008
(86) PCT No.: PCT/EP08/10666

§ 371 Date: Aug. 18, 2010

(30) Foreign Application Priority Data
Dec. 14, 2007 (GB) 0724393.4

(51) Int. Cl. A61K 31/56; C12Q 1/68; C40B 30/04; G01N 27/26

(52) U.S. Cl. 514/178; 435/6; 506/9; 204/456

(57) ABSTRACT

Fertility problems affect (1 in 10) couples in Western society, making it one of the most common serious health issues. Despite this, little is known about the causes of infertility, and thus patient counseling and treatment are suboptimal. With infertility being such a common problem, identification of any cause would impact on a large number of patients, allowing better counseling, clearer diagnoses and the possibility of making more informed choices (e.g. adoption vs. IVF treatment). The present invention provides methods to identify a cause of infertility in a subject based on the genotype of the subject, in particular, by evaluating the status of the gene encoding FK506 binding protein-like (FKBPL). In particular, the present invention relates to use of the status of the gene encoding FK506 binding protein-like for identification of a cause of an infertile phenotype in a subject. Also provided, are methods method for identifying an infertile phenotype in a subject, and identifying a cause of an infertile phenotype in a subject. This diagnostic tool finds wide clinical utility in the identification of a cause of infertility, resultantly impacting on a large number of patients. Further aspects of the present invention relate to the targeting of FKBPL in order to temporarily and reversibly induce infertility in a subject. Such aspects of the present invention find utility in the development of a male contraceptive pill. Moreover, due to the high degree of homology between the human and mouse FKBPL gene, FKBPL can be targeted in order to induce infertility in mice (or other species) as a form of pest control or animal husbandry.

### BACKGROUND

Fertility problems affect 1 in 10 couples in Western society, making it one of the most common serious health issues. Despite this, little is known about the causes of infertility, and thus patient counseling and treatment are suboptimal. The condition of infertility is multifactorial, with known causes of infertility including environmental factors, genetic alterations, and physical defects. Male infertility accounts for approximately half of the cases of reproductive failure in humans, with as many as 1 in 5 cases of male infertility having oligo- or azoospermia of unknown origin (Chandley et al., 1975). Since there are a number of possible causes, processing of cases can be time-consuming, and failure to identify the source of the problem can be very unsatisfactory for the couple. In about 15% of cases, moreover, the infertility investigation will show no abnormalities. In these cases abnormalities may be present but not detected by current methods. With recent advances in assisted reproduction techniques (ART), such as testicular sperm extraction (TSE) and intracytoplasmic sperm injection (ICSI) (Craft et al., 1993), successful conception can be achieved in more cases than ever before, increasing the urgency of identifying inherited factors responsible for infertility. Accordingly, it is desirable to provide a means to identify the causes of infertility, in particular in a subject where other diagnoses have failed.

Studies have indicated that microdeletions on the Y chromosome encompassing the DAZ or AZF genes are the cause of some types of azoospermia (Reijo et al., 1995; Vogt et al., 1992). Other known causes include mutations in the androgen receptor (AR) (Dowsing et al., 1999), important for sex hormone signaling, and in the synaptonemal complex protein 3 (SYCP3) (Miyamoto et al., 2003), a vital component of the structure which aligns chromosome pairs at meiosis, among others. However, altogether these still only account for a fraction of azoospermia cases, suggesting that other genetic causes remain to be discovered. In this connection, only a small number of genes have been identified as carrying mutations in infertile men. Previous studies have shown that targeted deletions of FKBP6 and FKBP52, members of the FK506 binding protein cochaperone family, cause male infertility in mice, but so far no mutations have been found in these genes in humans.

From studies of the role of the FKBP proteins, a number of possible functions for these cochaperones have been proposed. Some evidence suggests that they may alter receptor affinity for its cognate ligand, possibly by altering the folding of the ligand-binding domain (Riggs et al., 2003). Other work suggests that ligand binding is not affected in all cases, but that the stability of the receptor may be compromised in the absence of the cochaperone (CheungFlynn et al., 2005). A third point of action may be through involvement with nuclear transport. Studies have shown that FKBP52 binds to dynein, linking the receptor:HSP90 complex to the microtubule transport machinery (Periyasamy et al., 2007). Finally, other work suggests that FKBP52 may modulate activity of the androgen receptor (AR) through effects on transcription (CheungFlynn et al., 2005; Gallo et al., 2007; Yong et al., 2007), possibly by affecting coactivator recruitment.

FK506-binding protein-like (FKBPL) is a divergent member of the FKBP family of proteins, named for their ability to bind the immunosuppressant drug FK506. FKBPL belongs to that subfamily of FKBP, which act as cochaperones for steroid receptor complexes (Pratt and Toft, 2003). The ligand-binding domain (LBD) of these receptors undergoes a process of maturation, which is essential for their activation. This subfamily has two major domains: one that has a peptidylprolyl cis-trans isomerase (PPI) activity, and the other containing tetratricopeptide repeats (TPR). The FKBPL protein shows low homology over the PPI domain and lacks critical residues, which have been shown to be required for enzymatic activity (Kay, 1996), but is relatively well conserved at the TPR. The only known function of TPR is to avidly bind Heat Shock Protein 90 (HSP90) and the characterised TPR-containing members of the FKBP family have been shown to act as cochaperones with HSP90 to mediate steroid receptor folding and activation (Pratt and Toft, 2003). A dimer of the chaperone HSP90 binds to the receptor dimer and guides the folding, intracellular localisation and turnover of the protein, with the aid of cochaperones (Felts and Toft, 2003; Smith, 2004; Sullivan et al., 2002). FKBP bind HSP90 via conserved tetratricopeptide (TPR) repeats. The peptidylprolyl cis-trans isomerise (PPI) domain of the FKBP appears to be able to alter the folding of the LBD (Cheung-Flynn et al., 2005; Riggs et al., 2007). This domain also appears to be important for linking the chaperone:client protein complex to the cytoskeleton for inward transport to the nucleus: two groups have shown that FKBP52 binds to dynamitin, linking the receptor-HSP90 complex to the microtubule transport machinery (Galigniana et al., 2004; Periyasamy et al., 2007). The end result of FKBP-receptor interactions appears to be modulation of the activity of the receptor through effects on transcription (Cheung-Flynn et al., 2005; Yong et al., 2007; Gallo et al., 2007). The protein is highly conserved across several mammalian species, indicative of an important function. However, no mutations in FKBP52 (Beleza-Meireles et al., 2007) or FKBP6 (Westerveld et al., 2005) were found in azoospermic men.

There have been a number of targeted mutations generated in mice, which resulted in male infertility. In particular, mutations in two members of the FKBP family have resulted in male infertility. Mutations in FKBP52 were shown independently by two groups to result in male infertility and hypospadias with underdevelopment of the prostate and seminal vesicles, though the testes were normal (Cheung-Flynn et al., 2005; Yong et al., 2007). Both groups found evidence for compromised AR activity in the knockout mice, and showed that FKBP52 potentiates AR signalling in response to androgen. In a separate study, a spontaneous mutation in FKBP6 was found in an inbred rat strain which had developed azoospermia: the causative role of this mutation was shown using targeted deletion in mice, which resulted in the same phenotype (Crackower et al., 2003).

With infertility being such a common problem, identification of any cause would impact on a large number of patients, allowing better counseling, clearer diagnoses and the possibility of making more informed choices (e.g. adoption vs. IVF treatment).

Accordingly, it is an object of the present invention to identify a cause of infertility in a subject based on the genotype of the subject, in particular, by evaluating the status of the gene encoding FK506 binding protein-like (FKBPL).

For the purposes of this specification, the term “infertility” represents a medical condition attributable to factors such as genetic defects, wherein a subject is incapable of biological contribution to conception.

What is meant by the term “infertile phenotype” is the group of plastic characteristics, which manifest as an infertile state, and are affected by a combination of factors such as those relating to genetic traits, environmental influence, or anatomical defects.

### SUMMARY OF THE INVENTION

According to a first aspect of the present invention, there is provided the use of the status of the gene encoding FK506 binding protein-like for identification of a cause of an infertile phenotype in a subject.

Preferably, the subject is a human. Optionally, the subject is a male human. Alternatively, the subject is an animal. Preferably, the animal is a rodent. Optionally, the animal is a male rodent.

For the purposes of this specification, the term “gene status” is intended to refer to a multifactorial characteristic, which is determined by a combination of factors, such as qualitative or quantitative presence or absence of a wild-type gene, and/or qualitative or quantitative presence or absence of mutations in the gene; and/or qualitative or quantitative presence or absence of a transcription product such as RNA, and/or qualitative or quantitative presence or absence of a translation product such as a protein, and/or qualitative or quantitative presence or absence of a posttranslational modification such as addition of a functional group for activation.

Qualitative presence may be determined by a method for evaluating the presence of a gene, or an expression product thereof. For example, the presence of a gene, or an expression product thereof, above a detectable level may be indicative of the qualitative presence of the gene, or an expression product thereof. The detectable level may be based on the method chosen. Quantitative presence may be determined by a method for providing an indication of the amount of a gene, or an expression product thereof. Suitable methods for determination are described further herein.

It is envisaged that the present invention can be used to identify a cause of an infertile phenotype in a subject. However, it is understood that the subject does not necessarily display an infertile phenotype, and that the present invention can find utility in diagnosing any subject, regardless of the phenotype, with a cause of an infertile phenotype. Accordingly, the present invention also provides a method of identifying an infertile phenotype in a subject.

The term “diagnosis” is used herein to refer to the identification of a molecular or pathological state, disease or condition, such as the identification of a molecular subtype causing, or associated with, an infertile phenotype.

Preferably, the gene encoding FK506 binding protein like is FK506 binding protein like, as defined by Genbank Accession number AF139374, and Genbank Version number AF139374.1, GI:7707326; as disclosed by Robson et al. (1997) Gene regulation by low-dose ionizing radiation in a normal human lung epithelial cell line, Biochem. Soc. Trans. 25 (1), 335-342, which is incorporated in entirety herein by reference. The nomenclature “FK506 binding protein like” is intended to be synonymous with “FKBPL”,“DIR1”, or “NG7”.

Preferably, the gene comprises the nucleotide sequence depicted in SEQ ID NO. 1.

According to a second aspect of the present invention, there is provided a method for identifying an infertile phenotype in a subject, the method comprising the steps of identifying a subject with an atypical FKBPL gene status, and, optionally, attributing the infertile phenotype to the atypical FKBPL gene status.

According to a third aspect of the present invention, there is provided a method for identifying a cause of an infertile phenotype in a subject, the method comprising the steps of identifying a subject with an atypical FK506 binding protein-like gene status, and, optionally, attributing the cause of the infertile phenotype to the atypical FK506 binding protein-like gene status.

By the term “typical” is meant the status of a gene that is associated with a disease-free phenotype. In the present case, a typical FKBPL gene status may include presence of the FKBPL gene, optionally presence of the nucleic acid sequence depicted in SEQ ID N01. Alternatively or additionally, a typical FKBPL gene status may include presence of a polyribonucleotide transcribed from the FKBPL gene (optionally, from the nucleic acid sequence depicted in SEQ ID NO1), or a fragment thereof. Alternatively or additionally, a typical FKBPL gene status may include presence of a polypeptide encoded by the FKBPL gene (optionally, from the nucleic acid sequence depicted in SEQ ID NO1), or a fragment thereof.

By the term “atypical” is meant any deviation from a typical state, for example, any deviation from a typical gene status, including any alterations or variations to the gene that contribute to, cause, or are associated with, a disease setting. In the present case, an atypical FKBPL gene status may include absence of the FKBPL gene, optionally absence of the nucleic acid sequence depicted in SEQ ID NO1. Alternatively, in an atypical FKBPL gene status, the gene differs from the FKBPL gene, optionally differs from the nucleic acid sequence depicted in SEQ ID NO1. The difference may be at least one alteration or at least one variation in the structure or sequence of the gene.

It is understood that the alteration, or variation, to the gene may occur at the level of nucleic acid sequence, but encompassed within this definition, are any gene expression products including polyribonucleotides transcribed therefrom, and polypeptides translated from the said polyribonucleotides.

More specifically, an atypical FKBPL gene status may include absence of a polyribonucleotide transcribed from the FKBPL gene (optionally, from the nucleic acid sequence depicted in SEQ ID NO1), or a fragment thereof. Alternatively or additionally, an atypical FKBPL gene status may include absence of a polypeptide encoded by the FKBPL gene (optionally, from the nucleic acid sequence depicted in SEQ ID NO1), or a fragment thereof. Alternatively, in an atypical FKBPL gene status, the polyribonucleotide differs from the polyribonucleotide transcribed from the FKBPL gene (optionally, from the nucleic acid sequence depicted in SEQ ID NO1), or a fragment thereof. The difference may be at least one alteration or at least one variation in the structure or sequence of the polyribonucleotide. Alternatively, in an atypical FKBPL gene status, the polypeptide differs from the polypeptide encoded by the FKBPL gene (optionally, from the nucleic acid sequence depicted in SEQ ID NO1), or a fragment thereof. The difference may be at least one alteration or at least one variation in the structure or sequence of the polypeptide.

Optionally, the method may further comprise the step of obtaining a biological sample from a subject to permit identification of atypical FKBPL gene status in the subject. Optionally, the method may further comprise the step of isolating the FKBPL gene from the biological sample. Alternatively or additionally, the method may further comprise the step of evaluating the expression of the FKBPL gene in the biological sample.

Optionally, the biological sample is taken from a site wherein the FKBPL gene is expressed. Optionally, the biological sample is taken from the testes. Further optionally, the biological sample comprises cells of the tubule, or interstitial Leydig cells, of the testes.

Preferably, the status of the gene encoding FKBPL is evaluated by identifying at least one alteration, or at least one variation, to the FKBPL gene. More preferably, the status of the gene is evaluated by identifying alterations, or variations, to the gene depicted in SEQ ID N01.

The alterations, or variations, to the gene may result in dysfunctional FKBPL function. Dysfunctional FKBPL function may optionally affect the ability of FKBPL to bind to other biological entities, such as polypeptides or polynucleotides.

Optionally, the atypical FKBPL gene status is indicative of dysfunctional FKBPL function. Optionally, the atypical FKBPL gene status is indicative of azoospermia. Further optionally, the atypical FKBPL gene status is indicative of a lack of spermatogenesis. Alternatively, the atypical FKBPL gene status is indicative of oligozoospermia.

Optionally, the cause of an infertile phenotype is attributable to azoospermia. Further optionally, the cause of an infertile phenotype is attributable to a lack of spermatogenesis. Alternatively, the cause of an infertile phenotype is attributable to oligozoospermia.

Preferably, the status of the gene encoding FKBPL is evaluated using genomic-based approaches, such as PCR, Q-PCR, RFLP analysis, microarray, single-nucleotide primer extension (SNuPE), or single strand conformational polymorphism analysis (SSCP). Most preferably, the status of the gene encoding FKBPL is evaluated using nucleotide-sequencing techniques. However, any suitable approach may be utilised, which can be chosen by one skilled in the art.

Optionally, the alteration in the gene is a heterozygous alteration.

Further optionally, the at least one alteration in the gene comprises at least one mutation. Optionally, the at least one alteration in the gene is a single nucleotide polymorphism. The single nucleotide polymorphism may be a nonsynonymous single nucleotide polymorphism. Optionally, the single nucleotide polymorphism may be a missense nonsynonymous single nucleotide polymorphism. Further optionally, the single nucleotide polymorphism may be a nonsense nonsynonymous single nucleotide polymorphism.

Optionally, the single nucleotide polymorphism comprises a nucleotide substitution selected from the group comprising, but not limited to, C>C/T, G>C/G, and G>A/G.

By “C>C/T” is meant a nucleotide substitution resulting in two allelic versions of a gene, wherein the first allele has a cysteine residue at a given nucleotide position, and the second allele has a thymine residue at the same nucleotide position. By “G>C/G” is meant a nucleotide substitution resulting in two allelic versions of a gene, wherein the first allele has a cysteine residue at a given nucleotide position, and the second allele has a guanine residue at the same nucleotide position. By “G>A/G” is meant a nucleotide substitution resulting in two allelic versions of a gene, wherein the first allele has a adenine residue at a given nucleotide position, and the second allele has a guanine residue at the same nucleotide position.

Optionally, the single nucleotide polymorphism may be selected from the group represented by, but not limited to, rs35580488 and rs28732176. Further optionally, the single nucleotide polymorphism may be located at nucleotide position 3504588 or 3504624. The single nucleotide polymorphism represented by rs35580488 may be located at nucleotide position 3504588. Nucleotide positions on chromosome 6 are given relative the March 2006 human reference sequence (NCBI Build 36.1) produced by the International Human Genome Sequencing Consortium.

Further optionally, the single nucleotide polymorphism may be rs35580488.

Preferably, the at least one mutation is in the region encoding the peptidylprolyl cis-trans isomerase (PPI)-like domain of FKBPL. Optionally, the at least one mutation is in the region comprising nucleotide positions 32205086 to 32205430. The at least one mutation may also be in the region encoding a binding pocket region of FKBPL. Preferably, the at least one mutation is an insertion mutation. Optionally, the insertion mutation comprises the insertion of 12 nucleotides. Optionally, the insertion comprises the nucleic acid sequence TCTCATAAGTCT. However, it will be appreciated that any type of mutation, or any nucleic acid sequence insertion, in this region, which results in a loss-of-function, can also be applicable. More preferably, the mutation is in the region adjacent nucleotide position 968 of the gene. Nucleotide positions are given relative to the nucleotide positions depicted in SEQ ID N01.

Optionally or additionally, the mutation is in the region encoding a tetratricopeptide repeat within FKBPL. However, it will be appreciated that any type of mutation in this region, which results in a loss-of-function, can also be applicable. Optionally, the mutation in the region encoding a tetratricopeptide repeat within FKBPL, affects the ability of FKBPL to bind to HSP90 or p21. Further optionally, the mutation in the region encoding a tetratricopeptide repeat within FKBPL, affects the ability of FKBPL to bind to HSP90.

Alternatively and additionally, the mutation is a splice acceptor mutation. However, it will be appreciated that any type of mutation in this region, which results in a loss-of-function, can also be applicable. Preferably, the mutation is in the region adjacent nucleotide position 869 of the gene. Nucleotide position is given relative to the nucleotide positions designated in SEQ ID N01. Optionally, the mutation comprises a nucleotide substitution. Further optionally, the nucleotide substitution comprises substitution of an adenine nucleotide for a nucleotide selected from thymine, guanine, and cytosine. Preferably, the nucleotide substitution comprises substitution of an adenine nucleotide for a guanine nucleotide.

It is understood that the presence of more than one alteration or variation may occur in the same subject, or in the same biological sample. For example, a subject, or sample, may exhibit a single nucleotide polymorphism, and an insertion mutation or other alteration or variation as described herein.

Alternatively or additionally, the status of the gene encoding FKBLP may be evaluated by analysing FKBPL protein level. Optionally, the status of the gene encoding FKBPL may be evaluated by analysing factors such as FKBPL activity. Preferably, the level or activity of the FKBPL protein can be evaluated using proteomic-based approaches as described herein, such as amino acid sequencing, western blot analysis, or tissue microarray. Optionally, FKBPL protein levels, or activity, less than normal is indicative of azoospermia. Further optionally, FKBPL protein levels, or activity, less than normal is indicative of a lack of spermatogenesis. Alternatively, FKBPL protein levels, or activity, less than normal is indicative of oligozoospermia.

Optionally, FKBPL protein levels, or activity, in the range of about 0 to about 75% less than normal is indicative of azoospermia. Further optionally, FKBPL protein levels, or activity, in the range of about 0 to about 75% less than normal is indicative of a lack of spermatogenesis. Alternatively, FKBPL protein levels, or activity, in the range of about 0 to about 75% less than normal is indicative of oligozoospermia.

As used herein, the term “normal” is defined as a defined expression level of the FKBPL gene, the defined expression level being associated with a disease-free phenotype.

Alterations in the protein levels can be assessed using Western blotting, immunohistochemistry, immunofluorescence, or any other suitable approach chosen by one skilled in the art.

Ability of FKBPL to bind to other proteins such as HSP90, USP19, UIP28, androgen receptor, p21, p53 or dynamitin can be assessed using coimmunoprecipitation, GST-pulldown, in vitro complex assembly, competitive binding to immunoadsorbed protein, or any such suitable technique chosen by one skilled in the art.

Alternatively or additionally, the status of the gene encoding FKBLP may be evaluated by analysing the location of FKBPL protein. Optionally, at least 75% of the FKBPL protein being located outside the nucleus after treatment with R1881 is indicative of azoospermia. Further optionally, at least 75% of the FKBPL protein being located outside the nucleus is indicative of a lack of spermatogenesis. Alternatively, at least 75% of the FKBPL protein being located outside the nucleus is indicative of oligozoospermia.

Ability of FKBPL to bind to small molecules such as immunophilins (FK506, and related compounds), or ability of said small molecules to interfere with FKBPL-mediated effects on androgen receptors, can be assessed using binding assays or receptor reporter assays, or any such suitable technique chosen by one skilled in the art.

Ability of FKBPL, or mutated versions thereof, to modulate androgen receptor (AR) activity may be assessed using AR reporter assays in transfected cells, AR ligand binding assays, AR translocation assays, or AR stability assays as chosen by one skilled in the art. In the case of an AR non-responsive cell, it is envisaged that AR, or a functional equivalent, can be introduced into the AR non-responsive cell. Optionally, the AR is transfected into the AR non-responsive cell.

Optionally, the cause of an infertile phenotype in a subject may be identified in combination with the identification of other alterations, or variations, in genes such as SYCP3, USP9, Protamine; or other regions of the Y chromosome.

According to a fourth aspect of the present invention, there is provided a diagnostic kit for performing the method of identifying a cause of an infertile phenotype in a subject, the diagnostic kit comprising means for identifying an atypical FKBPL gene status in a subject, and optionally, instructions for attributing the cause of the infertile phenotype to the atypical FKBPL gene status.

According to a further aspect of the present invention, there is provided a method of treating a subject suffering from a disorder caused by or associated with dysfunctional steroid hormone receptor signalling, the method comprising altering of FKBPL activity.

By “dysfunctional steroid hormone receptor signalling” is meant alterations in steroid hormone receptor signalling events that result in a mutant phenotype, such as steroid hormone receptor activity or steroid hormone receptor localisation events. Such disorders caused by dysfunctional steroid hormone receptor signalling include androgen insensitivity syndrome, Reifenstein syndrome, AR-associated male infertility, AR-associated hypospadias, and Progesterone receptor-A receptor deficiency related female infertility.

Preferably, the steroid hormone receptor is an androgen receptor.

It is envisaged that in cases wherein the steroid hormone receptor signalling is decreased, the FKBPL activity is increased.

According to a still further aspect of the present invention, there is provided a method of inducing temporary infertility in a subject, the method comprising reversibly altering FKBPL activity.

It is envisaged that infertility is temporarily induced in a subject by reversibly decreasing FKBPL activity in the subject.

### BRIEF DESCRIPTIONS OF THE DRAWINGS

An embodiment of the present invention will now be described, by way of example with reference to the accompanying drawings, in which:

FIG. 1A is a schematic representation of the structure of human and mouse FKBPL genes;

FIG. 1B is a schematic representation of the structure of the polypeptides encoded by the human and mouse FKBP genes;

FIG. 1C is an alignment of the amino acid sequences of the PPI domain from FKBP6, FKBP12, and FKBPL;

FIG. 2A illustrates expression of the FKBP-like (Fkbpl) gene by screening of a normalised mouse multiple tissue expression (MTE) Multiple Tissue Array;

FIG. 2B is a Northern Blot analysis of testis mRNA showing transcription of the FKBPL gene during sexual maturation in the male mouse;

FIG. 3A is a DNA sequence trace depicting the nucleotide sequence of a region of the wild-type FKBPL gene, and a region of the FKBPL gene from an azoospermic patient that harbours an insertion mutation (boxed), which was not present in any of the controls;

FIG. 3B is a Western blot on human HT29 cells transfected with GFP-tagged FKBPL from patient 83 (p83) or a control (WT);

FIG. 4 illustrates FKBPL immunostaining carried out on human testis sections;

FIG. 5A is a graph depicting the effect of FKBPL, in the presence of the testosterone analogue R1881, on PSA activity in human prostate LNCaP cancer cells;

FIG. 5B is a graph depicting the effect of FKBPL from patient 83 on Androgen Receptor-mediated transcription in LNCaP cells in response to R1881;

FIG. 6 is a photograph depicting the translocation of FKBPL from the cytoplasm to the nucleus in response to dexamethasone treatment;

FIG. 7A is a schematic representation of the FKBPL gene indicating the location of a mutation at a splice acceptor site of the gene;

FIG. 7B is the nucleotide sequence of the splice acceptor site of the FKBPL gene; and

FIG. 7C is the nucleotide sequence of the splice acceptor site of the FKBPL gene of a patient harbouring a mutation within that region.

### MATERIALS AND METHODS

### Sequencing of FKBPL from Human Samples

The FKBPL gene was sequenced from 68 patient samples and 62 matched controls. The entire gene (including intron) was amplified by PCR using primers in the 5′ and 3′ flanks of the gene using standard techniques. PCR reactions were cleaned using Wizard columns (Promega) before direct sequencing using a Big Dye kit (Applied biosystems) and one of eight primers spaced throughout the gene to give overlapping reads, which were assembled into a contig. Sequencing reactions were cleaned over G50 autoseq columns (Amersham) before being run on an ABI prism 3100 sequencer. Patient samples showing mutations were cloned using TA cloning (Invitrogen) and individual clones sequenced to confirm the presence of the mutation.

Analysis of fkbpl mRNA Expression by RT-PCR

Testis tissue samples from different stages of spermatogenesis and other adult mouse tissues were collected for RNA analysis. Total RNA was extracted from the tissues using the RNeasy Mini Kit (Qiagen). First strand cDNA was synthesised from 1 μs total RNA in a 12.5 μl reaction mixture containing 10 mM Tris HCL (pH 8.3), 0.2 μg Oligo(dT)₁₅ primer (Promega), 1.5 mM deoxynucleoside triphosphates, 1×AMV-RT buffer and 7.5U AMV reverse transcriptase (Promega). Primers specific for fkbpl coding exon (mumsdirR TCCCAGCTCGAAACAGTTCT) and for 5′ exon (musdirf5ex CTTCCAGGCCTCAACATCAT) were used. PCR was performed in 25 μl containing, 1× Taq buffer, 200 μM each dNTPs, 0.4 μM each primer in a final concentration, 2U Taq (Invitrogen) and 1 μl cDNA. An initial deanaturation at 94° C. for 3 min was followed by 28 cycles of 45 seconds at 94° C., 1 minute at 61° C., and 1 minute at 72° C. followed by a final elongation step of 5 minutes at 72° C. For control reaction mouse β-actin was amplified using the following oligonucleotides (Bact1 GCTGTGCTATGTTGCTCTAGACTTC, Bact2 CTCAGTAACAGTCCGCCTAGAAGC) The PCR products were separated on a 1% agarose gel, a digital image captured using a Kodak digital camera.

### RNA Expression Using Human Multiple Tissue Expression Array and Mouse Master Blots

For tissue specific expression analysis premade Human multiple tissue expression array and Mouse master blots were used (BD biosciences). The Human MTE Array allows to screen for the presence and relative abundance of a gene transcript in a 75 fetal and adult tissues while Mouse Master Blot is normalised to provide semiquantitative data on tissue specificity and target mRNA abundance. The blots were probed by ³²P labelled DNA fragments (Highprime kit?). Human Fkbp6 probe was a 369 bp. length isolated PCR fragment amplified from Human genomic DNA using specific primers designed for the longest encoding exon (fkbp6hf CTTCACCTACCAACGAGGGG, fkbp6 hr AACCCTACAAAATACACAAAGCA). Mouse Fkbp probe was a purified PCR fragment amplified from mouse testis cDNA using primers (fkbp6mF ATGGACAAGCTTTCGATTCT, fkbp6mR CTGAAGATCTGCTTCCACAGG). Human and mouse Fkbpl probes were purified PCR fragment amplified from Human genomic DNA and mouse genomic DNA respectively. Specific primers were designed for the coding exon (8.6F CTAGG CTCCTGCTGCCGGCTACTG, 8.6R TCAGCAGTTGCTTTTTCCAGGTCC, MusdirR TCCCAGCTCGAAACAGTTCT, musdirF GAACGAGAAGAACAC CGCTC). Hybridisation and subsequent washes were carried out at 65° C. according to the method of Church and Gilbert (1984) (Church and Gilbert, 1984) at a probe concentration of 3×10⁶ counts/ml. Hybridised probe was detected by exposure of the washed membrane to X-ray film (Kodak) at −70° C. using with an intensifying screen.

### Nucleotide Sequence Screening of FKBPL Gene

Patient samples were obtained with informed consent and approved for screening in consultation with the Ethical Approval committees of the respective institutes. Infertile azoospermic patient and fertile male control groups were screened for variance in genomic DNA sequence within the FKBPL gene. Briefly, genomic DNA region harbouring the FKBPL gene was amplified using flanking primers 5′-GGCTCCAGGGTTAGTTGTCA-3′ and 5′-CCCAAATCTCACAGCACAGA-3′. Amplified DNA was purified with Wizard Gel PCR clean-up kit according to manufacturer's instructions (Promega Ltd, UK). PCR products were sequenced using a set of five primers 5′-AACCAGTCAGATGCCAGAGG-3′, 5′-CCTCTGGCATCTGACTGGTT-3′,5′-GAACCAGGTTCAGGTCAGC-3′, 5′-GACTAGCGAGAAGGAAGCC-3′ and 5′-GGCTTCCTTCTCGCTAGTC-3′ to cover the full region of the FKBPL gene using big dye terminator sequence kit (Applied Biosystems, UK) and ABI Prism 3130× sequence analyser. Sequences of patient and control samples were compared with reference sequence to detect mutations and SNP's using UCSC Human genome Blat service (http://genome.ucsc.edu/). Zygosity of mutations and SNP's was confirmed by sequencing TOPO-TA cloned PCR products.

### Expression of GFP-Tagged FKBPL

N-terminal GFP constructs were generated by cloning the coding sequence of WT and patient 83 (P83) FKBPL into pcDNA3.1/NT-TOPO-GFP plasmid (Invitrogen, UK), creating GFP-FKBPL-WT and GFP-FKBPL-P83 respectively. CHO cells were grown in DMEM supplemented with 10% FBS at 37oC. and 5% CO₂. Cells were seeded at 2×10⁵ cells per slide 24 hrs prior to transfection. Plasmids were transfected using lipofectamine as a carrier in serum free Opti-MEM media (Invitrogen, UK) for 6 hrs. Transfection mix was replaced by fresh media and cells were incubated for 24 hrs prior to analysis. Cell nuclei were visualised by Hoechst staining and GFP expression was analyzed using confocal microscopy. AR response to (over) expression of WT and P83 FKBPL was measured using a luciferase reporter assay. LNCaP cells were maintained in phenol red free RPMI1640 supplemented with 10% charcoal-dextran stripped FBS, and mM HEPES. Cells (4×10⁴ per well) were seeded in 24-well plate coated with fibronectin (Invitrogen™, UK). Cells were cotransfected with pPA6.1Luc reporter construct, GFP-FKBPL-WT or GFP-FKBPL-P83 or pcDNA3.1 empty vector control and pBIND Renilla plasmid for transfection efficiency correction. Transfection was carried out in serum/phenol red free RPMI1640. Six hours later, the transfection mix was replaced by normal media with or without 10 nM ligand R1881. AR transactivity was assessed 24 hrs post transfection using Dual-Glo luciferase Assay system (Promega, UK) according to manufacturer's instructions.

### Western Blot Analysis

Whole cell protein extracts were obtained by lysis of cells with protein extraction buffer (50 mM Tris-HCl, 150 mM NaCl, 5 mM EDTA, 10% glycerol and 1% Igepel), followed by centrifugation to remove cell debris. Protein extracts (30 ug) were fractionated on 7.5% SDS-polyacrylamide gel (Biorad, UK) and transferred to nitrocellulose membrane (Amersham, UK). Membranes were blocked for 1 hr at RT with 5% goat serum in Tris-buffered saline with 0.2% Tween-20 (TBST) prior to incubation with primary antibody (1:800) anti-FKBPL rabbit polyclonal IgG (PTG Labs, USA). After washing with TBST, membranes were exposed to horseradish peroxidise (HRP) labelled goat anti-rabbit IgG (1:2000) (sc-3837, Santa Cruz, USA). HRP activity was detected using ECL reagents (GE Healthcare, UK) according to manufacturer's instructions.

### Immunohistochemistry

Paraffin-embedded testis sections of a fertile human male were obtained from ProSci Inc, USA. Tissue was progressively rehydrated in water-ethanol solutions following dewaxing by xylene. Antigen retrieval was carried out by heating in 10 mM citrate buffer (pH 6.0) using a 600W microwave oven (2×3 min). Tissue was blocked with 10% goat serum in PBS-Tween20 (PBST) for 1 hr at RT followed by 15 min treatment with 1% hydrogen peroxide to remove endogenous peroxidise activity. Sections are washed twice with PBST. Tissue was incubated overnight with primary (1:100) Rabbit anti-human FKBPL IgG at 4° C., followed by 1 hr incubation with secondary (1:2000) goat anti-rabbit IgG-HRP. Control sections were mock incubated with primary antibody, followed by secondary antibody incubation under similar conditions. Immunostaining was carried out with DAB Substrate Plus kit (Zymed, USA) according to manufacturer's instructions. Tissues were counterstained for 1 min with hematoxylin (Sigma, UK), progressively dehydrated in ethanol, dipped in xylene and mounted in Mowiol (Calbiochem, UK).

### Nuclear Translocation

DU145 cells were seeded onto poly-L-lysine coated slides and grown in phenol red-free medium using charcoal stripped FBS for 24 hrs prior to irradiation. After 6 hrs, medium with or without ligand (dexamethasone) is added before allowing cells to recover for 24 hrs. Cells were then fixed in methanol. After rinsing in PBS, non-specific protein binding was blocked in PBS containing of 5% normal goat serum, 0.1% Bovine serum albumine (BSA), and 0.1% triton X-100 for 30 min. Sections were incubated at 4° C. in a humidified chamber overnight with a primary antibody, diluted 1:100 in PBS containing 1% BSA. On the following day, samples were thoroughly washed in PBS, after which the Alexa fluor 488 goat anti-rabbit secondary antibody (Molecular Probes) diluted 1:400 in 1% BSA and 0.1% triton X-100 in PBS was applied for 45 min. The samples were then washed in PBS again and mounted with Vectashield mounting media (Vector Laboratories, USA). Images were captured on a confocal microscope. Negative controls were obtained by replacing the primary antibody with normal goat serum.

### Androgen Receptor (AR) Activity

LNCaP cells were grown to near-confluence in phenol red-free media containing charcoal-stripped FBS before transfecting with Lipofectamine 2000 and DNA constructs according to the manufacturer's recommendations. After 6 hrs, medium with or without ligand (R1881) is added before allowing cells to recover for 24 hrs. Cells are lysed and assayed for reporter construct activity using the Stop and Glo kit (Invitrogen) as per manufacturer's instructions, with luciferase activity measured on a Tecan platereader.

### EXAMPLES

The following examples are described herein so as to provide those of ordinary skill in the art with a complete disclosure and description of the invention, and are intended to be purely exemplary of the present invention, and are not intended to limit the scope of the invention.

### Example 1

### FKBPL Structure and Expression

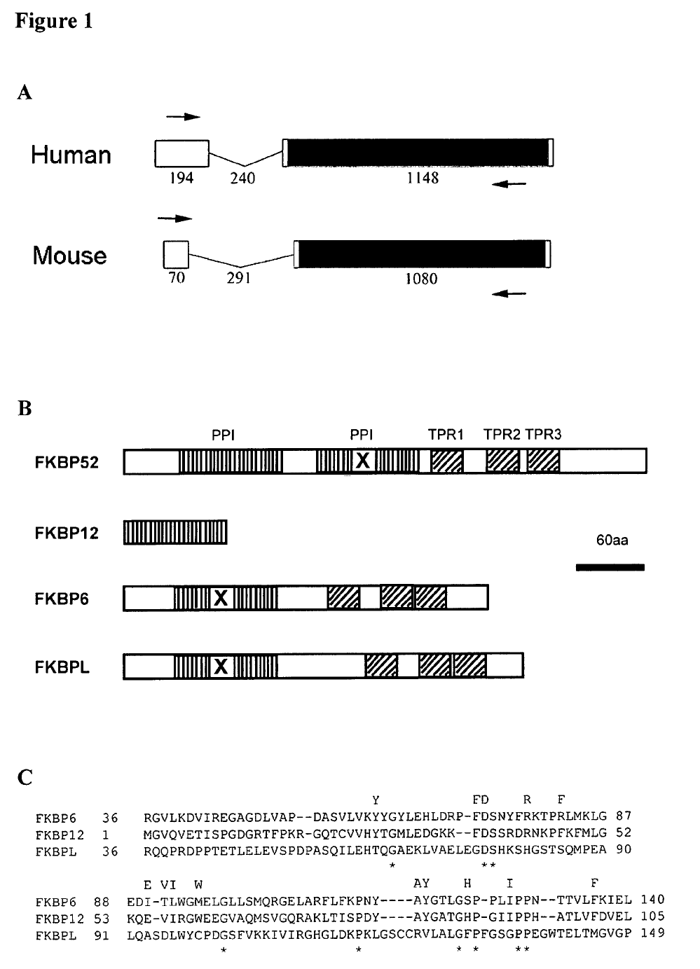

The FKBPL gene consists of two exons, joined by a short intron. As seen in FIG. 1A, the open reading frame is indicated in black, and the length is indicated in base pairs.

The protein is a divergent member of the FKBP family of proteins, named for their ability to bind the immunosuppressant drug FK506. FKBPL belongs to that subfamily of FKBP which act as cochaperones for steroid receptor complexes. Referring to FIG. 1B, this subfamily has two major domains, one of which has a peptidyl-prolyl cis-trans isomerase (PPI) activity and the other containing tetratricopeptide repeats (TPR). In FIG. 1B, Peptidyl-prolyl cis-trans isomerase (PPI) domains are shown by vertical shading, and tetratricopeptide repeats (TPR) are shown by diagonal shading. FKBP12 has a PPI domain but contains no TPR. FKBP52 and FKBP51 contain a duplication of the PPI domain, but the C-terminal copy is inactive (X). FKBP6 and FKBPL have N-terminal regions with some homology to the PPI.

The FKBPL protein shows low homology over the PPI domain and lacks critical residues, which have been shown to be required for enzymatic activity. In FIG. 1C, the residues conserved in the PPI with good enzymatic activity are indicated above the alignment but can be seen to be poorly conserved in FKBPL, but is relatively well conserved at the TPR. Conservative amino acid changes are underlined; identical residues are indicated by asterisks. The protein is highly conserved across several mammalian species, indicative of an important function.

### Example 2

### Fkbpl Transcription in Mouse

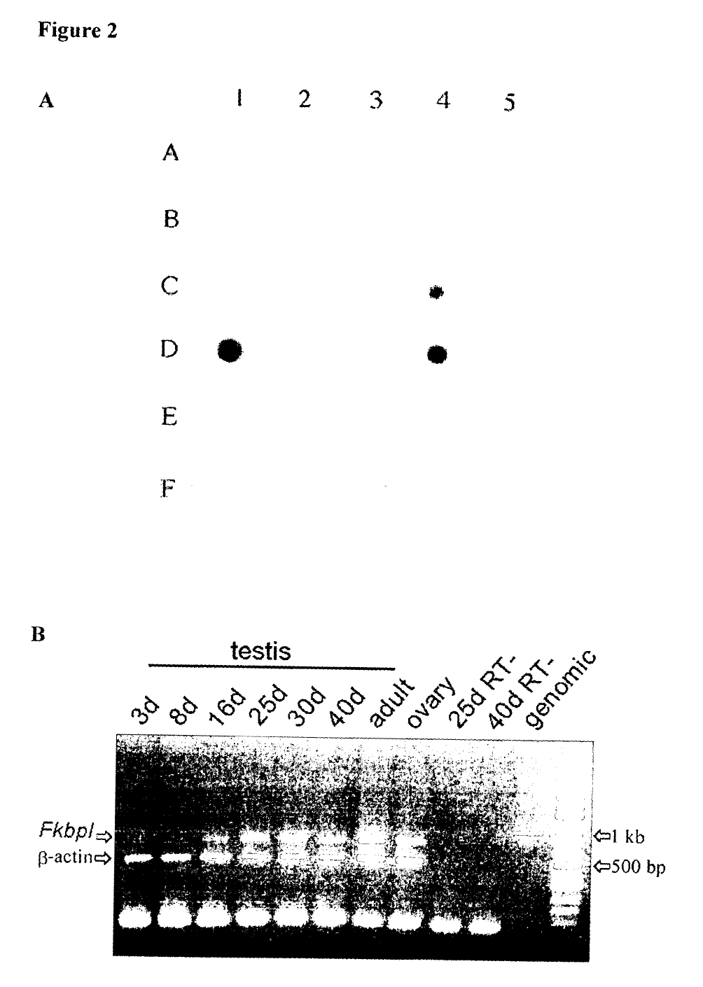

Referring to FIG. 2A, expression of the FKBP-like (Fkbpl) gene was examined by screening of a normalised mouse multiple tissue expression (MTE) Multiple Tissue Array mRNA blot hybridised to a radiolabelled Fkbpl cDNA, which shows high levels of transcription in testis (D1) and epididymus (D4). High levels of expression in submaxillary gland and low levels in all other tissues were confirmed by northern blotting (data not shown). RT-PCR of testis mRNA showed that transcription of the gene is turned on during sexual maturation in the male mouse at puberty (FIG. 2B). RT-PCR of total RNA isolated from testis at different days postnatally shows the appearance of transcripts as sexual maturation occurs. The primers span the intron, allowing the genomic product to be easily distinguished (right); b-actin is used as an internal control.

### Example 3

### FKBPL Expression in Human Testis

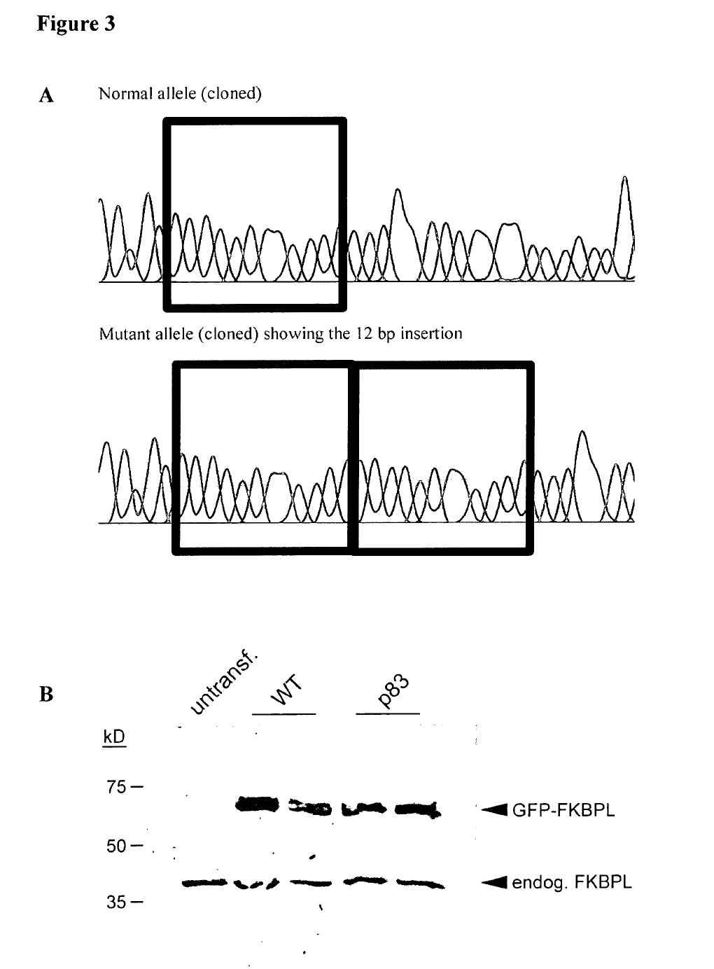

Expression in human tissues was widespread but was again strongest in testis by tissue array blot (data not shown). An antibody has been raised to FKBPL. In order to test its specificity we carried out western blots on human cell lines carrying a GFP-tagged version of the human protein (FIG. 3B). HT29 cells were transfected with GFP-tagged FKBPL from patient 83 (p83) or a control (WT). The size of the endogenous protein is also indicated. This result clearly showed that the antibody is picking up both endogenous and transfected FKBPL, with little or no background.

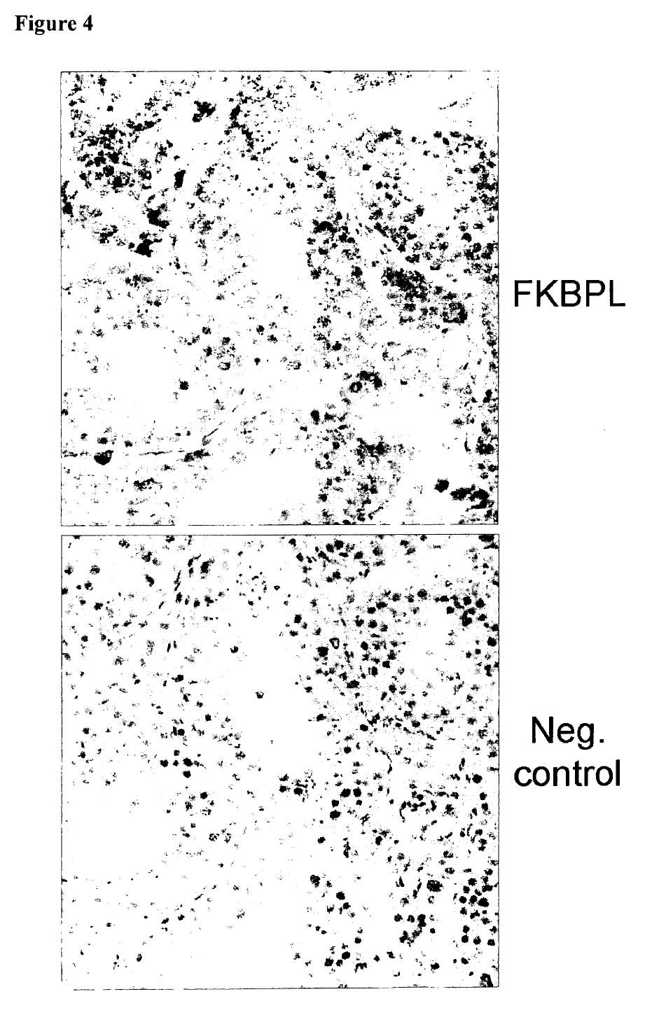

We then carried out immunostaining on human testis sections to see if FKBPL is expressed here (FIG. 4). The same antibody as in FIG. 3B shows staining (brown) in the spermatogonial cells of the tubule and the interstitial cells Leydig cells, but not in the cells of the tubule wall, peritubular myoid cells or blood vessels (blue). A secondary antibody control gave no non-specific background. This staining is very similar to that of FKBP52, another member of the same family, which has been shown to mediate Androgen Receptor (AR) activity (Cheung-Flynn et al., 2005). These data raise the possibility that FKBPL is involved in steroid hormone receptor signalling in the male reproductive organs.

### Example 4

### Azoospermia-Associated Mutations in FKBPL

FKBPL, a less well-characterised member of the FKBP family, has been shown to bind HSP90 through its TPR domain (Jascur et al., 2005) and (McKeen et al., 2008) have recently shown that it interacts with and stimulates the activity of glucocorticoid receptor (GR) in human cell lines. The fact that it interacts with GR and probably p53 is consistent with the behaviour of FKBP52, as shown by several groups (Cheung-Flynn et al., 2005; Yong et al., 2007; Galigniana et al., 2004) and with the model proposed by Pratt's group, which suggests that the cochaperone client protein is tissue-dependent. FKBPL maps to human chromosome 6p21.3: linkage studies in a Japanese population (Tsujimura et al., 2002) implicate this region specifically in azoospermia (LOD score 3.5, p=0.0005) and it also shows clustering of chromosomal breakpoints in azoospermic males in the European population (www.MCNdb.org).

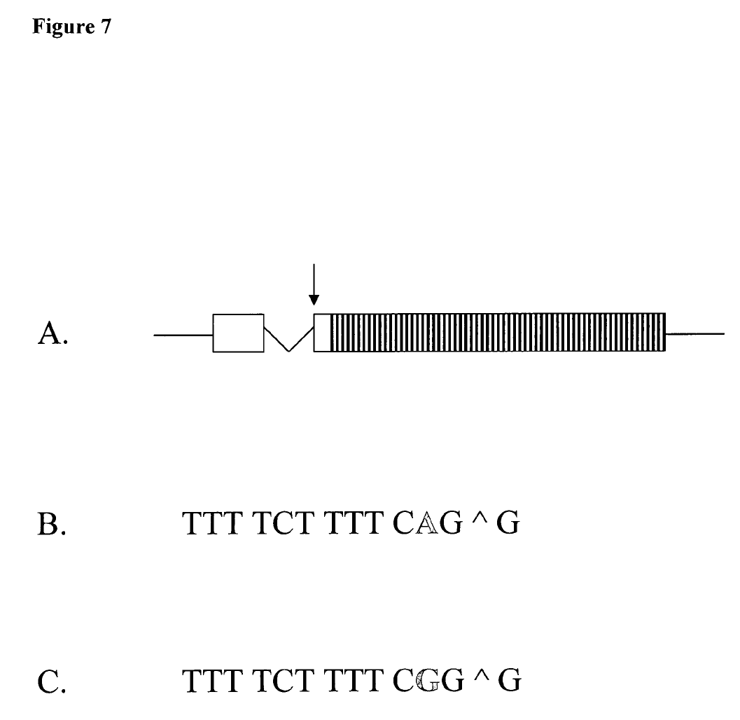

We found that FKBPL in humans maps to a region linked to azoospermia in a Japanese population (LOD score 3.5, p=0.0005) (Tsujimura et al., 2002). We examined 60 of the patient samples used in that study and 56 controls from the same population and looked for mutations in the FKBPL gene by direct sequencing. This identified two mutations in the gene in the azoospermic group: an insertion, which is predicted to alter a binding pocket (FIG. 3A), and a mutation in the canonical splice acceptor site (CAG/G->CGG/G) (FIG. 7), which is predicted to give loss of function. These were confirmed by sequencing individual clones and by sequencing of blinded samples at a second lab. Neither mutation was present in our control group. Some of the patient samples (14/60) also showed a different SNP pattern at some locations in the gene to those in the control group (Table 1), suggesting that these alterations may be significant or tightly linked to as-yet unidentified alteration elsewhere.

TABLE 1
SNP variation between Japanese patient and control group

| | DNA position: Chr6 | | | | | |
|---|---|---|---|---|---|---|
| | 1 | 2 | 3 | 4 | 5 | 6 |
| | @32205961 | @32205968 | @32205869 | @32205854 | @32205588 | @32205255 |
| Patients (n = 60) | 0 | 0 | 10 | 5 | 1 | 5 |
| Controls (n = 56) | 1 | 12 | 0 | 1 | 1 | 0 |

The table shows deviation from reference sequence for Patient group (top) vs Controls (bottom) at synonymous or non-coding sites mapped against sequence position. Coordinates are given with respect to the UCSC reference sequence for chromosome six.

Examination of the sixty patients from this cohort found a four amino acid insertion in the PPI domain in one patient, and a splice acceptor site mutation in another: both mutations were absent in a panel of fifty-six controls. The patient group SNP profile also differed from that of the control group (Table 1). For FKBPL, currently 14/60 patients have heterozygous alterations not seen so far in controls (23%), 2/60 are likely to be functional (3%). These data suggest that the mutations identified are associated with an azoospermic (or infertile) phenotype. This compares favourably with other mutations associated with male infertility: Y chromosome microdeletions are found in 2-20% of azoospermic males, depending on the study (Vogt et al., 1992); heterozygous mutations in SYCP3 which alter protein folding are found in 2/19 azoospermic males (11%) (Miyamoto et al., 2003); for USP9Y mutations, 17/576 patients showed alteration, but only 1 was de novo (3%-0.1%) (Sun et al., 1999), while for Protamine 1 (PRM1), heterozygous mutations were found in only 3/30 patients (10%) (Iguchi et al., 2006).

We also examined a second patient cohort of 30 patients from an Irish population where Y chromosome microdeletions have been excluded. Five of the patients had variations, which would alter the protein sequence of FKBPL (Table 2). The cohort from the Irish population showed two coding changes at SNPs in thirty azoospermic patients, which were not present in fertile controls.

TABLE 2
SNPs seen in the Irish azoospermic group

| | |
|---|---|
| Patient | Mutation/SNP |

| Nr | Type | Position | Mapped |
|---|---|---|---|
| 1 | C > C/T substitution | 3504457 | rs28732176 |
| | A > A/G substitution | 3505058 | rs204892 |
| 2 | A > A/G substitution | 3505058 | rs204892 |
| 3 | G > A/G substitution | 3504778 | rs41268905 |
| 4 | non | | |
| 5 | non | | |
| 6 | G > A/G substitution | 3504778 | rs41268905 |
| | A > A/G substitution | 3505058 | rs204892 |
| 7 | A > A/G substitution | 3505058 | rs204892 |
| 8 | non | | |
| 9 | C > C/T substitution | 3504457 | rs28732176 |
| | G > A/G substitution | 3505150 | rs9391734 |
| 10 | non | | |
| 11 | G > A/G substitution | 3504778 | rs41268905 |
| 12 | A > A/G substitution | 3505058 | rs204892 |
| 13 | non | | |
| 14 | A > A/G substitution | 3505058 | rs204892 |
| 15 | non | | |
| 16 | A > A/G substitution | 3505058 | rs204892 |
| 17 | non | | |
| 18 | | | |
| 19 | G > A/G substitution | 3504778 | rs41268905 |
| 20 | | 3505150 | Rs9391734 |
| 21 | non | | |
| 22 | G > C/G sustitution | 3504588 | rs35580488 |
| 23 | non | | |
| 24 | G > A/G substitution | 3504642 | NEW |
| 25 | G > A/G substitution | 3504778 | rs41268905 |
| 26 | non | | |
| 27 | non | | |
| 28 | A > A/G substitution | 3505058 | rs204892 |
| 29 | non | | |
| 30 | G > A/G substitution | 3505150 | rs9391734 |
| | C > C/T substitution | 3504457 | rs28732176 |

Missense mutations in CDS are displayed in bold.
Rs28732176: Alanine (non-polar, neutral) > Threonine (polar, neutral)
Rs35580488: Threonine (polar, neutral) > Arginine (polar, strongly basic)
NEW: Asparagine (polar, neutral) > Serine (polar, neutral)

### Example 5

### Effect of Steroid Hormone Receptor Ligand on Fkbpl Localisation

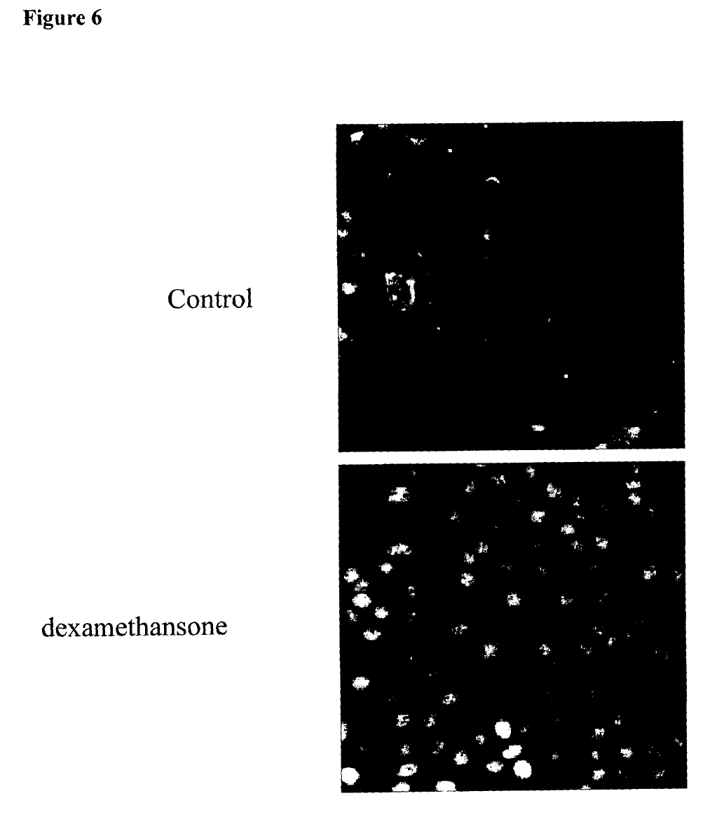

Jascur et al. have shown that FKBPL binds HSP90 through the TPR (Jascur et al., 2005). Data from our lab indicate that FKBPL translocates into the nucleus in response to stimulation of human cells with dexamethasone, a GR ligand (FIG. 6). These data confirm that FKBPL is found in HSP90:steroid receptor complexes and piggybacks on these complexes into the nucleus.

### Example 6

### Effect of FKBPL on Androgen Receptor Activity

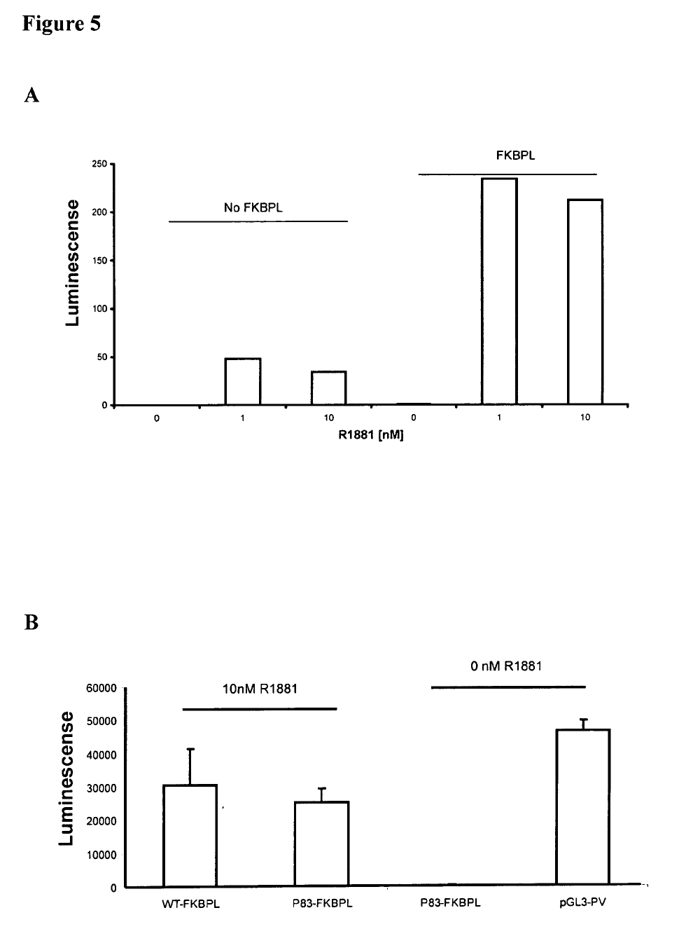

Given that our patients are azoospermic and infertile this suggested that FKBPL, like FKBP52, might be a cochaperone for Androgen Receptor in the testis. To check for AR interaction, we transfected LNCaP cells, an androgen-responsive prostate cancer cell line with high levels of AR, using a reporter construct (courtesy of Dr. J.-T. Liu) containing luciferase downstream of the Prostate Specific Antigen (PSA) transcriptional regulatory elements (Yong et al., 2007). To assess whether alterations in FKBPL affect AR function, the AR-positive prostate cell line, LNCaP, was transfected with a reporter containing luciferase driven by the prostate specific antigen (PSA) transcriptional regulatory elements. Referring to FIG. 5A, in the absence of the testosterone analogue R1881 little luciferase is detected. When ligand is added, transcription increases 50-fold due to AR action. With the addition of FKBPL, AR-mediated transcription increases instead 200-fold. FKBPL enhanced AR activity on the PSA reporter specifically in response to ligand (R1881). These results suggest that FKBPL enhances transcriptional activation by AR of a major target gene. Prostate cells expressing AR can turn on the prostate-specific antigen (PSA) (a known transcriptional target of the androgen receptor) reporter in the presence of a testosterone analogue (R1881).

Given that the splice acceptor site in patient 25 is predicted to prevent splicing into the only coding exon of the gene, AR activity in this patient is predicted to be suboptimal. To test whether the insertion seen in patient 83 is also functionally significant, we transfected LNCaP as above with a construct containing the cloned cDNA from this patient. A representative graph is shown in FIG. 5B. While enhancement of AR activity appeared lower in some experiments, results overall for this assay were inconclusive: it is possible however that the effects of this mutation may be stronger on AR target genes which are required for testis development. Mutant FKBPL, from a biological sample taken from patient 83 described above, shows decreased enhancement of Androgen Receptor-mediated transcription in LNCaP cells in response to ligand (left). PGL3-PV is a positive control (right). These data indicate that FKBPL does enhance the action of androgen receptor at a transcriptional level, and only in response to hormone, demonstrating a functional link between the androgen receptor and FKBPL.

Two members of the FKBP family of cochaperone proteins, FKBP52 and FKBP6, have previously been implicated in male sexual development in mice, but in case: control studies in humans no mutations were found in either gene in azoospermic males (Beleza-Meireles et al., 2007; Westerveld et al., 2005). FKBP6 has been reported to be a structural component of the synaptonemal complex and it is expressed at high levels in mouse testis (Crackower et al., 2003) but reprobing our array blots showed low levels of expression in epididymis and submaxillary gland (not shown), tissues where FKBPL levels were high. Expression in submaxillary gland is characteristic of steroid hormone signalling components (Jaskoll et al., 1994). FKBPL, like FKBP52 (Cheung-Flynn et al., 2005), was expressed in human testis in Leydig and Sertoli cells as well as spermatogonial cells: FKBP6, on the other hand, is absent from Sertoli cells (Crackower et al., 2003). Sertoli cells are AR-producing cells located inside the testis tubules where they play a crucial role in regulation of the spermatogonia. Cell type-specific knockout of AR in the Sertoli cells leads to azoospermia in mice, reinforcing the importance of AR for male sexual maturation (Chang et al., 2004; De Gendt et al., 2004; Wang et al., 2006). FKBP52 has been shown by two groups to act as a cochaperone for AR and to boost AR transcriptional activity in response to androgen (Cheung-Flynn et al., 2005; Yong et al., 2007). While the presence of FKBP52 in Sertoli and spermatogonial cells is consistent with a possible role in AR signalling, both groups found that gene knockouts in mice had no effect on the testis, but prostate and other secondary sexual organs expressing FKBP52 were reduced or absent. The expression pattern of FKBPL in human and mouse suggests a role in normal testis development which may be more similar to FKBP52 than FKBP6. Our data showing that transfection of FKBPL into the androgen responsive LNCaP cell line increases signalling through AR in response to ligand is consistent with this prediction.

FKBPL is a member of the TPR-containing subfamily of cochaperones and has been shown to bind to HSP90 via these repeats (Jascur et al., 2005). The PPI domain is poorly conserved and lacks conserved catalytic residues implicated in rotamase activity. Nevertheless, the protein is highly-conserved in mammals including across the PPI domain, suggesting a functional requirement. McKeen et al have recently shown that the PPI domain of FKBPL is important for interaction with dynamitin and subsequent nuclear translocation of steroid hormone:chaperone complex (McKeen et al., 2008). Pratt and coworkers have previously shown that the PPI domain of FKBP52 is also important for this interaction and for nuclear translocation in response to ligand (Galigniana et al., 2004). The splice acceptor mutation in patient 25 is predicted to prevent FKBPL production completely from this allele, which may reduce or abrogate entirely FKBPL protein levels in the cell if the protein is required for stability of a multimeric complex (Koi et al., 1994). The small insertion in patient 83 is predicted to alter a binding pocket in the PPI domain of the FKBPL protein, based on the crystal structure of FKBP52. Although not fully conclusive, our data suggest that the mutant FKBPL from this patient is also compromised in its ability to promote AR activity. FKBPL has also been reported to bind to p21 and enhance its stability (Jascur et al., 2005): however the mutant protein was able to coimmunoprecipitate p21 as efficiently as WT protein (data not shown). Other SNPs associated with azoospermia in our patient cohorts could be linked to more significant nearby alterations in regulatory regions or may have as-yet uncharacterised functional consequences.

The frequency of alterations in FKBPL in the azoospermic populations is low, but not inconsistent with the frequencies seen for other human genes implicated in infertility such as Y chromosome microdeletions (2-20% of infertile males (Vogt et al., 1992)); SYCP3 het. in 2/19 infertile males (Miyamoto et al., 2003)); USP9 (17/576 patients or 3% (Sun et al., 1999)) and Protamine 1 het. in 3/30 patients (10% (Iguchi et al., 2006)). A large number of genes will contribute to normal fertility so it is to be expected that the individual contributions of any one gene will be low, especially if autosomal. The heterozygous nature of the mutations uncovered so far, a feature of other genes implicated in human infertility (above) could indicate haploinsufficiency, or that the mutation on the other chromosome is as yet undetected due to a distal location. It is also of note that the FKBP6 gene in humans has been reported to be monoallelically expressed (Zhang et al., 2007). Further studies will be required to determine the frequency of mutations in the gene in other populations and to elaborate the possible functions of the protein.

In summary, FKBPL is a member of a cochaperone family which enhance steroid hormone receptor signalling and our data show that it is expressed in testis in human and mouse and that it is highly conserved. We have found mutations in the gene in azoospermic infertile patients, and have shown that the wild-type protein can enhance AR signalling in an androgen-responsive cell line, and AR is known to be crucial for male fertility.

Taken together these data suggest that FKBPL mediates the ligand-induced transcriptional activity of steroid hormone receptors, possibly by facilitating transport of ligand:receptor complexes from the cytoplasm to the nucleus. Furthermore, these data suggest that certain mutations in the FKBPL gene, which may result in atypical FKBPL activity, are associated with azoospermia. Accordingly, the present invention provides a means of determining such FKBPL gene alterations for the identification of a cause of infertility in a subject.

This diagnostic tool finds wide clinical utility in the identification of a cause of infertility, resultantly impacting on a large number of patients. By identifying causes of infertility, the present invention allows for the opportunity to offer patients better counseling, clearer diagnoses and the possibility of making more informed choices (e.g. adoption vs. IVF treatment).

Further aspects of the present invention relate to the targeting of FKBPL in order to temporarily and reversibly induce infertility in a subject. Such aspects of the present invention find utility in the development of a male contraceptive pill. Moreover, due to the high degree of homology between the human and mouse FKBPL gene, FKBPL can be targeted in order to induce infertility in mice (or other species) as a form of pest control or animal husbandry.

### REFERENCES

Beleza-Meireles, A., Barbaro, M., Wedell, A., Tohonen, V. and Nordenskjold, A. 2007. Studies of a co-chaperone of the androgen receptor, FKBP52, as candidate for hypospadias. Reprod. Biol. Endocrinol. 5: 8.
Chandley, A. C., Edmond, P., Christie, S., Gowans, L., Fletcher, J., Frackiewicz, A. and Newton, M. 1975. Cytogenetics and infertility in man. I. Karyotype and seminal analysis: results of a five-year survey of men attending a subfertility clinic. Ann. Hum. Genet. 39: 231-254.
Chang, C., Chen, Y. T., Yeh, S. D., Xu, Q., Wang, R. S., Guillou, F., Lardy, H. and Yeh, S. 2004. Infertility with defective spermatogenesis and hypotestosteronemia in male mice lacking the androgen receptor in Sertoli cells. Proc. Natl. Acad. Sci. U.S.A. 101: 6876-6881.
Cheung-Flynn, J., Prapapanich, V., Cox, M. B., Riggs, D. L., Suarez-Quian, C. and Smith, D. F. 2005. Physiological role for the cochaperone FKBP52 in androgen receptor signaling. Mol. Endocrinol. 19: 1654-1666.
Church, G. M. and Gilbert, W. 1984. Genomic sequencing. Proc. Natl. Acad. Sci. U.S.A. 81: 1991-1995.
Crackower, M. A., Kolas, N. K., Noguchi, J., Sarao, R., Kikuchi, K., Kaneko, H., Kobayashi, E., Kawai, Y., Kozieradzki, I., Landers, R., Mo, R., Hui, C. C., Nieves, E., Cohen, P. E., Osborne, L. R., Wada, T., Kunieda, T., Moens, P. B. and Penninger, J. M. 2003. Essential role of Fkbp6 in male fertility and homologous chromosome pairing in meiosis. Science 300: 1291-1295.
Craft, I., Bennett, V. and Nicholson, N. 1993. Fertilising ability of testicular spermatozoa. Lancet 342: 864.
De Gendt, K., Swinnen, J. V., Saunders, P. T., Schoonjans, L., Dewerchin, M., Devos, A., Tan, K., Atanassova, N., Claessens, F., Lecureuil, C., Heyns, W., Carmeliet, P., Guillou, F., Sharpe, R. M. and Verhoeven, G. 2004. A Sertoli cell-selective knockout of the androgen receptor causes spermatogenic arrest in meiosis. Proc. Natl. Acad. Sci. U.S.A. 101: 1327-1332.
Dowsing, A. T., Yong, E. L., Clark, M., McLachlan, R. I., de Kretser, D. M. and Trounson, A. O. 1999. Linkage between male infertility and trinucleotide repeat expansion in the androgen-receptor gene. Lancet 354: 640-643.
Felts, S. J. and Toft, D. O. 2003. P23, a Simple Protein with Complex Activities. Cell Stress Chaperones 8: 108-113.
Galigniana, M. D., Harrell, J. M., O'Hagen, H. M., Ljungman, M. and Pratt, W. B. 2004. Hsp90-binding immunophilins link p53 to dynein during p53 transport to the nucleus. J. Biol. Chem. 279: 22483-22489.
Gallo, L. I., Ghini, A. A., Pilipuk, G. P. and Galigniana, M. D. 2007. Differential Recruitment of Tetratricorpeptide Repeat Domain Immunophilins to the Mineralocorticoid Receptor Influences both Heat-Shock Protein 90-Dependent Retrotransport and Hormone-Dependent Transcriptional Activity. Biochemistry
Iguchi, N., Yang, S., Lamb, D. J. and Hecht, N. B. 2006. An SNP in protamine 1: a possible genetic cause of male infertility? J. Med. Genet. 43: 382-384.
Jascur, T., Brickner, H., Salles-Passador, I., Barbier, V., El Khissiin, A., Smith, B., Fotedar, R. and Fotedar, A. 2005. Regulation of p21 (WAF1/CIP1) stability by WISp39, a Hsp90 binding TPR protein. Mol. Cell 17: 237-249.
Jaskoll, T., Choy, H. A. and Melnick, M. 1994. Glucocorticoids, TGF-beta, and embryonic mouse salivary gland morphogenesis. J. Craniofac. Genet. Dev. Biol. 14: 217-230.
Kay, J. E. 1996. Structure-function relationships in the FK506-binding protein (FKBP) family of peptidylprolyl cis-trans isomerases. Biochem. J. 314 (Pt 2): 361-385.
Koi, M., Umar, A., Chauhan, D. P., Cheman, S. P., Carethers, J. M., Kunkel, T. A. and Boland, C. R. 1994. Human chromosome 3 corrects mismatch repair deficiency and microsatellite instability and reduces N-methyl-N′-nitro-N-nitrosoguanidine tolerance in colon tumor cells with homozygous hMLH1 mutation. Cancer Res. 54: 4308-4312.
McKeen, H. D., McAlpine, K., Valentine, A., Quinn, D. J., McClelland, K., Byrne, C., O'Rourke, M., Young, S., Scott, C. J., McCarthy, H. O., Hirst, D. G. and Robson, T. 2008. A novel FK506-like binding protein interacts with the glucocorticoid receptor and regulates steroid receptor signaling. Endocrinology 149: 5724-5734.
Miyamoto, T., Hasuike, S., Yogev, L., Maduro, M. R., Ishikawa, M., Westphal, H. and Lamb, D. J. 2003. Azoospermia in patients heterozygous for a mutation in SYCP3. Lancet 362: 1714-1719.
Periyasamy, S., Warrier, M., Tillekeratne, M. P., Shou, W. and Sanchez, E. R. 2007. The immunophilin ligands cyclosporin A and FK506 suppress prostate cancer cell growth by androgen receptor-dependent and -independent mechanisms. Endocrinology 148: 4716-4726.
Pratt, W. B. and Toft, D. O. 2003. Regulation of signaling protein function and trafficking by the hsp90/hsp70-based chaperone machinery. Exp. Biol. Med. (Maywood) 228: 111-133.
Reijo, R., Lee, T. Y., Salo, P., Alagappan, R., Brown, L. G., Rosenberg, M., Rozen, S., Jaffe, T., Straus, D. and Hovatta, 0.1995. Diverse spermatogenic defects in humans caused by Y chromosome deletions encompassing a novel RNA-binding protein gene. Nat. Genet. 10: 383-393.
Riggs, D. L., Cox, M. B., Tardif, H. L., Hessling, M., Buchner, J. and Smith, D. F. 2007. Non-Catalytic Role of the Fkbp52 Peptidyl-Prolyl Isomerase Domain in the Regulation of Steroid Hormone Signaling. Mol. Cell. Biol.
Smith, D. F. 2004. Tetratricopeptide repeat cochaperones in steroid receptor complexes. Cell Stress Chaperones 9: 109-121.
Sullivan, W. P., Owen, B. A. and Toft, D. O. 2002. The influence of ATP and p23 on the conformation of hsp90. J. Biol. Chem. 277: 45942-45948.
Sun, C., Skaletsky, H., Birren, B., Devon, K., Tang, Z., Silber, S., Oates, R. and Page, D. C. 1999. An azoospermic man with a de novo point mutation in the Y-chromosomal gene USP9Y. Nat. Genet. 23: 429-432.
Tsujimura, A., Ota, M., Katsuyama, Y., Sada, M., Miura, H., Matsumiya, K., Gotoh, R., Nakatani, T., Okuyama, A. and Takahara, S. 2002. Susceptibility gene for non-obstructive azoospermia located near HLA-DR and -DQ loci in the HLA class II region. Hum. Genet. 110: 192-197.
Vogt, P., Chandley, A. C., Hargreave, T. B., Keil, R., Ma, K. and Sharkey, A. 1992. Microdeletions in interval 6 of the Y chromosome of males with idiopathic sterility point to disruption of AZF, a human spermatogenesis gene. Hum. Genet. 89: 491-496.
Wang, R. S., Yeh, S., Chen, L. M., Lin, H. Y., Zhang, C., Ni, J., Wu, C. C., di Sant'Agnese, P. A., deMesy-Bentley, K. L., Tzeng, C. R. and Chang, C. 2006. Androgen receptor in sertoli cell is essential for germ cell nursery and junctional complex formation in mouse testes. Endocrinology 147: 5624-5633.
Westerveld, G. H., Repping, S., Lombardi, M. P. and van der Veen, F. 2005. Mutations in the chromosome pairing gene FKBP6 are not a common cause of non-obstructive azoospermia. Mol. Hum. Reprod. 11: 673-675.
Yong, W., Yang, Z., Periyasamy, S., Chen, H., Yucel, S., Li, W., Lin, L. Y., Wolf, I. M., Cohn, M. J., Baskin, L. S., Sanchez, E. R. and Shou, W. 2007. Essential role for Co-chaperone Fkbp52 but not Fkbp51 in androgen receptor-mediated signaling and physiology. J. Biol. Chem. 282: 5026-5036.
Zhang, W., Zhang, S., Xiao, C., Yang, Y. and Zhoucun, A. 2007. Mutation screening of the FKBP6 gene and its association study with spermatogenic impairment in idiopathic infertile men. Reproduction 133: 511-516.

1-30. (canceled)

31. A method for identifying a cause of an infertile phenotype in a subject, the method comprising the steps of identifying a subject with an atypical FK506 binding protein-like gene status, and attributing the cause of the infertile phenotype to the atypical FK506 binding protein-like gene status.

32. The method according to claim 31, wherein the atypical FK506 binding protein-like gene sequence differs from the nucleotide sequence depicted in SEQ ID NO. 1.

33. The method according to claim 31, wherein the method further comprises the step of evaluating the expression of the FK506 binding protein-like gene in the biological sample.

34. The method according to claim 31, wherein the status of the gene is evaluated by identifying alterations to the gene.

35. The method according to claim 34, wherein the alteration to the gene comprises a single nucleotide polymorphism comprising a nucleotide substitution selected from C>C/T, G>C/G, and G>A/G.

36. The method according to claim 35, wherein the single nucleotide polymorphism is selected from rs35580488 and rs28732176.

37. The method according to claim 35, wherein the single nucleotide polymorphism is located at nucleotide position 3504624 of human chromosome 6.

38. The method according to claim 34, wherein the alteration to the gene is a mutation in the region encoding the peptidylprolyl cis-trans isomerase (PPI)-like domain of FKBPL.

39. The method according to claim 38, wherein the mutation is in the region adjacent nucleotide position 968 of the gene.

40. The method according to claim 38, wherein the mutation is an insertion mutation.

41. The method according to claim 38, wherein the mutation comprises the insertion of twelve nucleotides.

42. The method according to claim 38, wherein the mutation comprises the insertion of the nucleic acid sequence TCTCATAAGTCT.

43. The method according to claim 38, wherein the mutation comprises the insertion of twelve nucleotides at nucleotide position 968 of the gene.

44. The method according to claim 38, wherein the mutation is a splice acceptor mutation.

45. The method according to claim 38, wherein the mutation is in the region adjacent nucleotide position 869 of the gene.

46. The method according to claim 38, wherein the mutation comprises a nucleotide substitution.

47. The method according to claim 46, wherein an adenine nucleotide is substituted with a nucleotide selected from thymine, guanine, and cytosine.

48. The method according to claim 46, wherein an adenine nucleotide is substituted with a guanine nucleotide.

49. The method according to claim 31, wherein FK506 binding protein-like gene status is evaluated by analysing an expression product of the FK506 binding protein-like gene.

50. The method according to claim 49, wherein the expression product is FKBPL protein.

51. The method according to claim 31, wherein the cause of an infertile phenotype in a subject is identified in combination with the identification of other alterations in genes selected from SYCP3, USPS, and Protamine.

52. A diagnostic kit for identifying a cause of an infertile phenotype in a subject, the diagnostic kit comprising means for identifying an atypical FKBPL gene status in a subject, and instructions for attributing the infertile phenotype to the atypical FKBPL gene status.

53. A method of treating a subject suffering from a disorder caused by or associated with dysfunctional steroid hormone receptor signalling, the method comprising altering FKBPL activity.

54. The method according to claim 53, wherein the steroid hormone receptor is an androgen receptor.

55. The method according to claim 53, wherein FKBPL activity is increased.

56. A method of inducing temporary infertility in a subject, the method comprising reversibly altering FKBPL activity.

57. The method according to claim 56, wherein FKBPL activity is decreased.


SEQUENCE LISTING (26 sequences)

SEQ ID NO: 1 - DNA, 1357 bp, Homo sapiens
gggttagttgtcagtatctttcccagttgttccgccccctacccccgcctcccgcaccgcgcccctctccggctgccctctccgcgtggggcaaggctccgagggcagcattcagtagccatttagctttggaaggagaggtgattcgaatggcccggctcctcctgtcaccatgctaggcactttggccgcgcaggtgctgacctgaacctggttcatccctttctgaccaaaactgttcactcaccgtggaagggactaagcatccatatggagacgccaccagtcaatacaattggagaaaaggacacctctcagccgcaacaagagtgggaaaagaaccttcgggagaaccttgattcagttattcagattaggcagcagccccgagaccctcctaccgaaacgcttgagctggaagtaagcccagatccagccagccaaattctagagcatactcaaggagctgaaaaactggttgctgaacttgaaggagactctcataagtctcatggatcaaccagtcagatgccagaggcccttcaagcttctgatctctggtactgccccgatgggagctttgtcaagaagatcgtaatccgtggccatggcttggacaaacccaaactaggctcctgctgccgggtactggctttggggtttcctttcggatcagggccgccagagggctggacagagctaactatgggcgtagggccatggagggaggaaacttggggggagctcatagagaaatgcttggagtccatgtgtcaaggtgaggaagcagagcttcagctgcctgggcactctggacctcctgtcaggctcacactggcatccttcactcaaggccgagactcctgggagctggagactagcgagaaggaagccctggccagggaagaacgtgcaaggggcacagaactatttcgagctgggaaccctgaaggagctgcccgatgctatggacgggctcttcggctgctcctgactttacccccacctggccctccagaacgaactgtccttcatgccaatctggctgcctgtcagttgttgctagggcagcctcagttggcagcccagagctgtgaccgggtgttggagcgggagcctggccatttaaaggccttataccgaaggggggttgcccaggctgcccttgggaacctggaaaaagcaactgctgacctcaagaaggtgctggcgatagatcccaaaaaccgggcagcccaggaggaactggggaaggtggtcattcaggggaagaaccaggatgcagggctggctcagggtctgcgcaagatgtttggctgattaaaagttaaaccttaaaagagaaaaaaaaaaaaaa

SEQ ID NO: 2 - DNA, 12 bp, Homo sapiens
tctcataagtct

SEQ ID NO: 3 - DNA, 20 bp, Artificial Sequence
  Synthetic Primer Sequence
tcccagctcgaaacagttct

SEQ ID NO: 4 - DNA, 20 bp, Artificial Sequence
  Synthetic Primer Sequence
cttccaggcctcaacatcat

SEQ ID NO: 5 - DNA, 25 bp, Artificial Sequence
  Synthetic Primer Sequence
gctgtgctatgttgctctagacttc

SEQ ID NO: 6 - DNA, 24 bp, Artificial Sequence
  Synthetic Primer Seuence
ctcagtaacagtccgcctagaagc

SEQ ID NO: 7 - DNA, 20 bp, Artificial Sequence
  Synthetic Primer Sequence
cttcacctaccaacgagggg

SEQ ID NO: 8 - DNA, 23 bp, Artificial Sequence
  Synthetic Primer Sequence
aaccctacaaaatacacaaagca

SEQ ID NO: 9 - DNA, 20 bp, Artificial Sequence
  Synthetic Primer Sequence
atggacaagctttcgattct

SEQ ID NO: 10 - DNA, 21 bp, Artificial Sequence
  Synthetic Primer Sequence
ctgaagatctgcttccacagg

SEQ ID NO: 11 - DNA, 24 bp, Artificial Sequence
  Synthetic Primer Sequence
ctaggctcctgctgccggctactg

SEQ ID NO: 12 - DNA, 24 bp, Artificial Sequence
  Synthetic Primer Sequence
tcagcagttgctttttccaggtcc

SEQ ID NO: 13 - DNA, 20 bp, Artificial Sequence
  Synthetic Primer Sequence
tcccagctcgaaacagttct

SEQ ID NO: 14 - DNA, 20 bp, Artificial Sequence
  Synthetic Primer Sequence
gaacgagaagaacaccgctc

SEQ ID NO: 15 - DNA, 20 bp, Artificial Sequence
  Synthetic Primer Sequence
ggctccagggttagttgtca

SEQ ID NO: 16 - DNA, 20 bp, Artificial Sequence
  Synthetic Primer Sequence
cccaaatctcacagcacaga

SEQ ID NO: 17 - DNA, 20 bp, Artificial Sequence
  Synthetic Primer Sequence
aaccagtcagatgccagagg

SEQ ID NO: 18 - DNA, 20 bp, Artificial Sequence
  Synthetic Primer Sequence
cctctggcatctgactggtt

SEQ ID NO: 19 - DNA, 19 bp, Artificial Sequence
  Synthetic Primer Sequence
gaaccaggttcaggtcagc

SEQ ID NO: 20 - DNA, 19 bp, Artificial Sequence
  Synthetic Primer Sequence
gactagcgagaaggaagcc

SEQ ID NO: 21 - DNA, 19 bp, Artificial Sequence
  Synthetic Primer Sequence
ggcttccttctcgctagtc

SEQ ID NO: 22 - protein, 104 aa, Homo sapiens
RGVLKDVIREGAGDLVAPDASVLVKYYGYLEHLDRPFDSNYFRKTPRLMKLGEDITLWGMELGLLSMQRGELARFLFKPNYAYGTLGSPPLIPPNTTVLFKIEL

SEQ ID NO: 23 - protein, 104 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVEL

SEQ ID NO: 24 - protein, 115 aa, Homo sapiens
RQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGP

SEQ ID NO: 25 - DNA, 13 bp, Homo sapiens
ttttcttttcagg

SEQ ID NO: 26 - DNA, 13 bp, Homo sapiens
ttttcttttcggg